(19) United States
(12) Patent Application Publication
(10) Pub. No.: US 2005/0101007 A1
(43) Pub. Date: May 12, 2005

(54) Specimen testing device

(76) Inventor: Garry Tsaur, Rowland Heights, CA (US)

Correspondence Address: JOE NIEH, 18760 E. AMAR ROAD #204, WALNUT, CA, 91789, US

(21) Appl. No.: 10/704,925

(22) Filed: Nov. 10, 2003

(51) Int. Cl. C12M 1/34

(52) U.S. Cl. 435/287.2

(57) ABSTRACT

An integrated specimen collecting and testing device comprising a hollow elongated tubular housing with a sealed end and another sealed end with an opening means is disclosed. An indicator strip is disposed within the sealed elongated tubular housing wherein a specimen may enter the hollow elongated tubular housing after the opening means is opened and comes in contact with the indicator strip thereby the property of the specimen may be determined by visual inspection of the indicator strip.

### BACKGROUND

1. Field of Invention

The present invention relates generally to a device for collecting and testing specimen. More specifically the present invention relates to a sealed specimen collector for collecting and testing liquid specimen.

2. Description of Related Art

Specimen often needs to be collected and tested. The specimen may be colleted for acidity tests and for pregnancy tests. A technician or the subject himself may perform the collection of specimen. The specimen may be collected by using a spatula, a bulb and pipette, or a syringe to obtain the specimen and then placing the specimen in a separate container. The specimen may also be collected by using a vacuum container with preexisting vacuum in the container to suck the specimen into the container to be collected. However, the vacuum container has a limited shelf life due to the slow leakage of air into the vacuum container. Furthermore, a vacuum container generally has a relatively large volume to allow a reasonable amount of specimen to be collected even after some reduction of vacuum due to the leakage of air into the vacuum container.

The collected specimen is then sent to a laboratory for testing. The collected specimen may also be tested at or near the collection site with a portable testing and indicating device such as a test strip that may be place in contact with the specimen to indicate the property of the specimen with a change of color of the test strip or other visual indicators. The test strip must be stored in a separately sealed container and must be disposed of after use. The specimen and the container used to hold the specimen must also be disposed of after use.

### SUMMARY OF THE INVENTION

The present invention is an integrated specimen collecting and testing device comprising a hollow elongated tubular housing with a sealed end and another sealed end with an opening means. An indicator strip is disposed within the sealed elongated tubular housing. After opening the sealed hollow elongated tubular housing through the opening means, the hollow elongated tubular housing is squeezed to force the air out of the hollow elongated housing. The opened end of the hollow elongated tubular housing is then placed in contact with the specimen to be collected and tested and the pressure is released from the hollow elongated tubular housing to allow it to return to its original shape wherein it will draw in the specimen through the opened end. The specimen will enter the hollow elongated tubular housing and comes in contact with the indicator strip wherein the property of the specimen may be determined by visual inspection of the indicator strip.

### BRIEF DESCRIPTION OF THE DRAWINGS

FIG. 1 shows the preferred embodiment of the specimen testing device.

FIG. 2 shows another embodiment of the specimen testing device.

### DETAILED DESCRIPTION OF THE PREFERRED EMBODIMENT

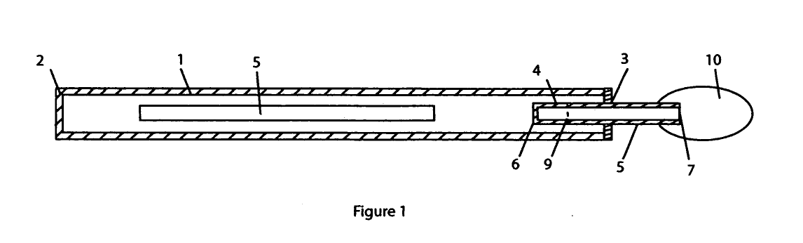

FIG. 1 shows the preferred embodiment of the present invention. In the preferred embodiment, the specimen testing device comprises of a hollow elongated tubular housing 1 with a first sealed end 2 and a second sealed end 3 with an opening means 4 provided near the second sealed end 3. An indicator strip 5 is disposed within the hollow elongated tubular housing 1. Prior to opening the hollow elongated tubular housing 1, the indicator strip 5 is sealed in the enclosed environment of the hollow elongated tubular housing 1.

The preferred opening means 4 comprises of a hollow tube 5 with a sealed end 6 and an open end 7 with a score line 9 formed near the sealed end 6 that will allow the hollow tube 5 to be broken open at the score line 9 when bent at or near the score line 9. The hollow tube 5 is inserted with the sealed end 6 and the score line 9 inside the end 3 of the hollow elongated tubular housing 1 thereby sealing the end 3 of the hollow elongated tubular housing 1. The outside diameter of the hollow tube 5 near the sealed end 3 is predetermined such that when it is inserted into the end 3 of the hollow elongated tubular housing 1, it will completely seal the end 3 of the hollow elongated tubular housing 1 yet still allow the seal end 6 of the hollow tube 5 to be severed when the hollow tube 5 and the hollow elongated tubular housing 1 are bent at or near the score line 9 thereby resulting in a fluid flow path from the open end 7 of the hollow tube 5 to the interior of the hollow elongated tubular housing 1. An optional swab or foam tip 10 may be affixed to the open end 7 of the hollow tube 5 to act as an absorber and filter of the specimen.

After opening the sealed hollow elongated tubular housing 1 through the opening means 4, the hollow elongated tubular housing 1 is squeezed to force the air out of the hollow elongated housing 1. The opened end of the hollow elongated tubular housing 1 is then placed in contact with the specimen to be collected and tested and the pressure is released from the hollow elongated tubular housing 1 to allow it to return to its original shape wherein it will draw in the specimen through the opened end. The specimen will enter the hollow elongated tubular housing 1 and comes in contact with the indicator strip 5 wherein the property of the specimen may be determined by visual inspection of the indicator strip 5. After testing, the entire device may then be disposed of sanitarily.

Although the description above contains many specificities, these should not be construed as limiting the scope of the invention but as merely providing illustrations of some of the presently preferred embodiments of this invention. Thus the scope of the invention should be determined by the appended claims and their legal equivalents, rather than by the examples given.

1. A specimen testing device comprising:
a hollow elongated tubular housing with a first sealed end and a second sealed end;
an opening means provided near said second sealed end of said hollow elongated tubular housing; and
an indicator strip is disposed within the hollow elongated tubular housing;
wherein after opening said hollow elongated tubular housing through the opening means, the hollow elongated tubular housing may be used to collect the specimen through the opened end for testing with the indicator strip.

2. A specimen testing device as in claim 1, wherein said opening means further comprises a hollow tube with a sealed end and an open end with a score line formed near the sealed end that will allow the hollow tube to be broken open at the score line when bent at or near the score line whereby the hollow tube is inserted with the sealed end and the score line inside the end of the hollow elongated tubular housing thereby sealing the end of the hollow elongated tubular housing wherein the outside diameter of the hollow tube near the sealed end is predetermined such that when it is inserted into the end of the hollow elongated tubular housing it will completely seal the end of the hollow elongated tubular housing yet still allow the seal end of the hollow tube to be severed when the hollow tube and the hollow elongated tubular housing are bent at or near the score line thereby resulting in a fluid flow path from the open end of the hollow tube to the indicator strip.

3. A specimen testing device as in claim 1, wherein a swab or foam tip is affixed to the open end of the hollow tube to act as an absorber and filter of the specimen.

4. A specimen testing device as in claim 2, wherein a swab or foam tip is affixed to the open end of the hollow tube to act as an absorber and filter of the specimen.
